SEQUENCE LISTING (2 sequences)

SEQ ID NO: 1 - protein, 448 aa, Homo sapiens
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Homo sapiens
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0051458 A1
(43) Pub. Date: Feb. 13, 2025

(54) Methods for Treating Neurodegeneration Targeting IGF1/IGF1R

(71) Applicant: The Schepens Eye Research Institute, Inc., Boston, MA (US)

(72) Inventor: Dong Feng Chen, Newtonville, MA (US)

(21) Appl. No.: 18/566,166

(22) PCT Filed: May 31, 2022
(86) PCT No.: PCT/US2022/031615

§ 371 Date: Dec. 1, 2023

Related U.S. Application Data
(60) Provisional application No. 63/195,546, filed on Jun. 1, 2021.

(51) Int. Cl. C07K 16/28; A61K 31/58; A61K 38/30; A61P 25/28; A61P 27/02

(52) U.S. Cl. CPC C07K 16/2863; A61K 31/58; A61K 38/30; A61P 25/28; A61P 27/02; C07K 2317/24

(57) ABSTRACT

Described are methods for the treatment of disorders associated with microglial activation, including neurodegenerative diseases and immunomodulatory diseases, particularly immunomodulatory diseases of the eye. Generally, the methods include administering a therapeutically effective amount of an IGF1 or IGF1R antibody or antigen binding fragment thereof, or an IGF1R agonist, as described herein, to a subject who is in need of, or who has been determined to be in need of, such treatment.

### INCORPORATION BY REFERENCE

This application claims priority to U.S. Provisional Patent Application No. 63/195,546, filed on Jun. 1, 2021. The disclosure of the prior application is considered part of the disclosure of this application, and is incorporated herein by reference in its entirety.

### STATEMENT REGARDING FEDERAL FUNDING

This invention was made with government support under Grant No. EY025259 awarded by the National Institutes of Health. The government has certain rights in the invention.

### SEQUENCE LISTING

This application contains a Sequence Listing that has been submitted electronically as an ASCII text file named 00633-0326W01_ST25.txt. The ASCII text file, created on May 31, 2022, is 5.91 kilobytes in size. The material in the ASCII text file is hereby incorporated by reference in its entirety.

### TECHNICAL FIELD

Described are methods for the treatment of disorders associated with microglial activation, including neurodegenerative diseases and immunomodulatory diseases, particularly immunomodulatory diseases of the eye, by administering a therapeutically effective amount of an IGF1 or IGF1R antibody or antigen binding fragment thereof, or an IGF1R agonist, as described herein, to a subject who is in need of, or who has been determined to be in need of, such treatment.

### BACKGROUND

Microglia are resident macrophage-like immune cells in the central nervous system. Accumulating evidence indicates that they not only play import roles in inflammatory diseases, such as multiple sclerosis and uveitis, but have emerged as key players in many neurodegenerative diseases, including Alzheimer's Disease, amyotrophic lateral sclerosis (ALS), Parkinson's Disease, stroke, brain or spinal cord trauma or ischemia, glaucoma, ischemic and traumatic optic neuropathy, non-arteritic anterior ischemic optic neuropathy (NAION), age-related macular degeneration, diabetic retinopathy, autism and other diseases^{1, 2}. In addition, tissue-resident macrophages play important roles in maintaining tissue homeostasis and innate immune defense against invading microbial pathogens. Clinical observations of dry eye and Sjogren's syndrome have associated these conditions with dysregulated immune homeostasis and increased inflammatory responses.

### SUMMARY

Provided herein methods and compositions for treating a neurodegenerative disease in a subject, or for treating an immunomodulatory disease in a subject, e.g., in an eye or the central nervous system (CNS; e.g. brain and spinal cord) of a subject. Also provided are IGF1 or IGF1R antibody or antigen binding fragment thereof, or an IGF1R agonist, for use in a method of treating a neurodegenerative disease in a subject, and for use in a method for treating an immunomodulatory disease in a subject, e.g., in an eye or CNS of a subject. The methods comprise administering a therapeutically effective amount of an IGF1 or IGF1R antibody or antigen binding fragment thereof, or an IGF1R agonist.

In some embodiments, the neurodegenerative disease is Alzheimer's Disease, Parkinson's Disease, amyotrophic lateral sclerosis (ALS), stroke, brain and spinal cord trauma or ischemia, glaucoma, ischemic and traumatic optic neuropathy, Non-arteritic anterior ischemic optic neuropathy (NAION), optic neuritis, age-related macular degeneration, macular edema, or diabetic retinopathy.

In some embodiments, the immunomodulatory disease in a subject, e.g., in an eye of a subject, is anterior uveitis, dry eye, or Sjogren's syndrome, or is ocular inflammation as a result of ocular disease or procedure, for example, corneal transplantation.

In some embodiments, the methods include administering 3G5C1 antibody or antigen-binding fragments thereof, or a humanized version thereof.

In some embodiments, the methods include administering an IGF1R agonist, e.g., a small molecule agonist, e.g., RG5.

In some embodiments, the disease is in an eye of the subject, and the method comprises systematic administration (e.g. intra-vein, intra peritoneal, intramuscular, or subcutaneous injection or oral) administration to the affected eye.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-D. Increased inflammatory gene markers of retinal microglia from IGFBPL1 deficiency mice. (A) quantification of the number of activated microglia in the whole-mounted retina from WT and Igfbpl1^{-/-} mice at the age of 1-, 2- and 7-month-old; qPCR results showed the expression level of activated microglia markers (B) and pro-inflammatory cytokines (C) were up-regulated in 7 m old Igfbpl1^{-/-} mice retina. Data are from n=4 for each group. (D) Dot plot of representative expression markers on resident microglia. Top row: markers of common microglia including CX3CR1; middle row: markers of cluster 1 inflammatory Mg with expressing MICII, Cd74, Apoe and Igf1 as well; bottom row, markers of cluster 2 typical genes of IFN response pathway and cluster 3 proliferating maker gene Ki67.

FIGS. 2A-C. IGFBPL1 deficiency caused neuroinflammation and progressive neurodegeneration in brain. Quantification the number of activated microglia (A) with increased size of cell body (B) and shorten process length (C) from WT and Igfbpl1^{-/-} mice. Data are from n=4 mice per group. Error bar=SEM. *P<0.05; **P<0.01; ***P<0.001.

FIGS. 3A-E. IGFBPL1 is required for preventing pTau and Aβ accumulation in the mouse brain. Representative Western blots (A) and quantification (B-E) of pTau, Aβ, APP and caspase-3 in the hippocampus of 7-month-old WT and IBKO mice (n=3 mice/group). Dots represent individual data. Data are mean±SEM.

FIGS. 4A-H. IGFBPL1 suppressed the activation of microglia and astrocyte induced by glaucoma in mice. Representative images of Brn3a+ RGCs (A) and Iba-1+ microglia cells (B) Quantification of RGCs numbers in WT and Igfbpl1^{-/-} mice retina with different ages. Data are from n=4 for each group. (D) Representative images of Brn3a+ RGCs and Iba-1+ microglia cells in the whole-mounted retina derived from mice with normal retina, MB+Saline and MB+IGFBPL-1 treatments. Bar=50 um (C) Quantification of RGC density in normal retinas and the retinas derived from mice received sham treatment, MB+saline and MB+IGFBPL1 (100 ng) at 8 weeks post-anterior chamber injection. Data are from n=7 mice per group. (E) and (F) presented the quantifications of total microglia and activated microglia density in glaucoma mice with Saline or IGFBPL1 given. RT-PCR assay showing the relative expression of (G) activated microglia markers and (H) pro-inflammatory cytokines in normal mice, and mice with sham treatment, MB+saline and MB+IGFBPL1 at 14 days post-MB injection. Data are from n=5 mice per group. Error bar=SEM. Bar in (A)=50 um. *P<0.05; **P<0.01; ***P<0.001.

FIGS. 5A-L. Therapeutic administration of IGFBPL1 prevents retinal neuron loss and rescues visual function in different mouse models of glaucoma via signaling IGF-1R in microglia. Graphs showing (A) contrast sensitivity (CS) and (B) visual acuity (VA) of optomotor response in mice received anterior chamber injection of MB and intravitreal injections of saline or IGFBPL1. Data are from n=10 mice per group. (C) Graphs showed pSTR response of ERG in mice received microbead injection and intravitreal injections of saline or IGFBPL1. The relative response is represented by the amplitude of ERG response of the injected eye (saline or IGFBPL1) in each mouse. Representative waveform (D) and amplitude quantifications (E) of pSTR response of ERG in DBA2J mice at different timepoints. Data are from n=8 mice in PBS group and n=9 in IGFBPL1 group. (I) representative phase contrast images at 1,000× magnification of cross-sections of optic nerve. Bar in (G)=10 um. The quantifications of survived RGC numbers (F) and axons (H) in 10 m-old DBA/2J mice with PSB or IGFBPL1 given, comparing with 6 m-old naïve DBA/2J mice as control. Data are from n=8 mice in PBS and IGFBPL1 group, and n=5 in control group. Contrast sensitivity (J) and visual acuity (K) measurement and pSTR responses of ERG performance (L) in experimental glaucoma mouse model using Igf-1r f/f littermate control and Igf-1r conditional knock out on microglia mice with PBS or IGFBPL1 intravitreal injection. (I) The quantification of RGC survival number in the whole-mounted retina of Igf-1r f/f littermate control and Igf-1r conditional knock out on microglia mice with PBS or IGFBPL1 intravitreal injection. Error bar=SEM. *P<0.05; ***P<0.001

FIGS. 6A-H. IGFBPL1 protects the degeneration of RGC, retinal function and spatial vision of a chronic glaucoma model of DBA/2J mice. (A) Showing the IOP profile of mice received sham treatment (green line), anterior chamber injection of microbead (MB) and intravitreal injections (blue arrows) of saline (red line) or IGFBPL1 (blue line) at Day 3, 10 and 17 post-injection of MB. Note the IOP level was measured non-invasively by TonoLab. Data are from n=10 mice per group. (B) IOP profile of DBA/2J mice received monthly intravitreal injections of PBS (black solid line) and BPL1 (red solid line) starting from 6-month-old age. Data are from n=8 mice in PBS group and n=9 in IGFBPL1 group. (C) Representative photomicrographs showing Brn3a+ RGCs (red) in flat-mount retinas derived from DBA/2J mice with PBS and IGFBPL-1 treatments, and (E) quantification of axon density in optic nerve of DBA/2J mice received intravitreal injections of PBS and BPL1. Data are from n=8 mice per group. IOP profile (D), pSTR responses of ERG performance (F), contrast sensitivity (G), and visual acuity (H) measurements in littermate sham mice or experimental glaucoma mouse model using Igf-1r f/f littermate control and Igf-1r conditional knock out on microglia mice with PBS or IGFBPL1 intravitreal injection. The quantification of RGC survival number in the whole-mounted retina of sham mice or experimental glaucoma Igf-1r f/f littermate control and Igf-1r conditional knock out on microglia mice with PBS or IGFBPL1 intravitreal injection. Error bar=SEM. *P<0.05; **P<0.01 and ***P<0.001.

FIG. 7. Screening for anti-human IGF1R agonist antibodies that has a counter-inflammatory effect in LPS-primed human microglial cell line HMC3. Result of qPCR measuring levels of pro-inflammatory cytokine IL-6, IL-1b and IFNa induction in HMC3 microglial cell line that were cultured alone (Ctrl), stimulated by LPS (100 ng/ml) and ATP (5 mM; LPS+ATP) following by treatment with vehicle (PBS), recombinant IGFBPL1 (BPL1) or various IGF1R antibodies at 6 hours after administration of LPS+ATP. Note the potent anti-inflammatory effect of Abnova anti-IGF1R antibody, but not other (R&D and Novus) antibodies, as assessed by all 3 pro-inflammatory cytokine mRNAs. Error bar=SEM. BPL1 was used as a positive control. *P<0.05 by one way ANOVA (n=4/group).

FIG. 8. Screening for anti-human IGF1 agonist antibody with a counter-inflammatory effect on microglial activation. Result of qPCR measuring levels of pro-inflammatory cytokine IL-6 induction in human microglial cell line that were stimulated by LPS and ATP (LPS+ATP) followed by treatment with IGF1R agonist Rg5 or various IGF1 antibodies 6 hours after LTP+ATP administration. IGF1R agonist Rg5 showed the greatest anti-inflammatory effect. Error bar=SEM. *P<0.05 by one way ANOVA.

FIG. 9. Rescue of RGC function and vision by intravitreal injection of IGF1R agonist in a mouse model of ischemic optic neuropathy. Quantifications of electroretinogram positive scotopic threshold response (pSTR) amplitudes, visual acuity and contrast sensitivity before (Baseline) and at 4 weeks (4 wks) after induction of ischemic optic neuropathy in mice received intravitreal injections of control or RG5 IGF1R agonist at day 1, 8 and 17 post injury. Error bar=SEM. *P<0.05 by one way ANOVA.

### DETAILED DESCRIPTION

Microglia exist in resting and activated states. In the healthy brain and retina, they play critical roles in performing constant surveillance, defense and cell support^{3, 4}. Following injury or insults, microglia lose their homeostatic molecular signature and function and become activated. Uncontrolled or sustained microglial activation and neuroinflammation are common phenomena observed in many neurodegenerative diseases;^{5, 6} thus, it is imperative that activated microglia are returned to a resting state once the insult or injury is resolved to prevent excessive inflammation. To date, the molecular signals that mediate the transition between the resting and activated states of microglia, especially that drives inflammatory microglia to homeostasis and support their house-keeping functions, are yet to be defined.

As demonstrated herein, insulin-like growth factor binding protein like protein 1 (IGFBPL1) is an essential counter-regulator of microglial activation in vitro and in vivo, and it does so by binding to IGF1 and forming a complex with IGF1R to mediate microglia gene network and functions (Guo et al, Scientific Reports 8:2054 (2018)). Moreover, we discovered that this effect of IGFBPL1 can be achieved by specific IGF1R agonist or IGF1R agonist antibody which mimics the activities of IGFBPL1-IGF1-IGF1R complex. Therapeutic administration of selective IGF1R agonist or IGF-1R antibodies presented potent and lasting anti-inflammatory and neuroprotective effects and functional benefits when examined in mouse models of glaucoma and ischemic optic neuropathy.

In addition, the present finding of a role of IGFBPL1 in microglia suggests that IGFBPL1 may also present strong anti-inflammatory effect on activated/inflammatory macrophages by reversing their inflammatory profile without affecting the homeostatic macrophages. Thus, IGFBPL1 and specific IGF1R agonist and IGF1R agonist antibodies that mimic the activities of IGFBPL1-IGF1-IGF1R complex can also be used for treating corneal inflammatory diseases, include anterior uveitis, dry eye and Sjogren's syndrome.

### Methods of Treatment

The methods described herein include methods for the treatment of disorders associated with microglial activation, including neurodegenerative diseases and immunomodulatory diseases, particularly immunomodulatory diseases of the eye. In some embodiments, the neurodegenerative disease is Alzheimer's Disease, Parkinson's Disease, amyotrophic lateral sclerosis (ALS), multiple sclerosis, autism, stroke, brain and spinal cord trauma or ischemia, glaucoma, ischemic and traumatic optic neuropathy, Non-arteritic anterior ischemic optic neuropathy (NAION), optic neuritis, age-related macular degeneration, macular edema, or diabetic retinopathy. In some embodiments, the immunomodulatory disease in a subject, e.g., in an eye of a subject, is uveitis, dry eye, or Sjogren's syndrome. In some embodiments, the disease is not glaucoma, but ocular inflammation as a result of ocular disease or procedure, for example, corneal transplantation. Generally, the methods include administering a therapeutically effective amount of an IGF1 or IGF1R antibody or antigen binding fragment thereof, or an IGF1R agonist, as described herein, to a subject who is in need of, or who has been determined to be in need of, such treatment.

As used in this context, to “treat” means to ameliorate at least one symptom of the disorder associated with microglial activation. Often, microglial activation results in inflammation leading to neuronal dysfunction and/or neuronal cell death; thus, a treatment (comprising administration of a therapeutically effective amount of a compound described herein) can result in a reduction in neuronal dysfunction and/or neuronal cell death, and a reduction in one or more symptoms of a neurodegenerative disease or immunomodulatory disease in the subject.

Provided herein are methods for treating neurodegenerative diseases using IGF1 and IGF1R antibodies or antigen binding fragments thereof, and for treating immunomodulatory disease using IGF1R agonists, and IGF1 and IGF1R antibodies as described herein.

### IGF1 and IGF1R Antibodies

In some embodiments, the methods include administration of one or more IGF1 and IGF1R antibodies. The term “antibody” as used herein refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, nonhuman, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dubel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dubel, Handbook of Therapeutic Antibodies: Technologies, Emerging Developments and Approved Therapeutics, (Wiley-VCH; 1 edition Sep. 7, 2010).

In some embodiments, the antibody is the 3G5C1 antibody or antigen-binding fragments thereof. The 3G5C1 antibody was raised against a recombinant protein corresponding to amino acids 1101-1367 of IGF-IR of human origin, and is commercially available from a number of sources including Abnova, Santa Cruz Biotechnology, and LSBio, among others. In some embodiments, the antibodies are humanized.

In some embodiments, the antibody is the teprotumumab antibody or antigen-binding fragments thereof. Teprotumumab is a 150 kDa fully human monoclonal antibody that targets the insulin-like growth factor receptor (IGFR1). Teprotumumab binds to the cysteine-rich region of the IGFR1 extracellular domain with high-affinity and specificity. Teprotumumab is sold as Tepezza in the United States by Horizon Therapeutics USA. Teprotumumab is disclosed in WO2005005635, which is incorporated by reference in its entirety, and has a heavy chain and light chain as identified below.

Heavy chain teprotumumab
(SEQ ID NO: 1)
QVELVESGGGVVQPGRSQRLSCAASGFTFSSYGMHWVRQAPGKGLEWVA
IIWFDGSSTYYADSVRGRFTISRDNSKNTLYLQMNSLRAEDTAVYFCAR
ELGRRYFDLWGRGTLVSVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
Light chain teprotumumab
(SEQ ID NO: 2)
EIVLTQSPATLSLSPGERATLSCRASQSVSSYLAWYQQKPGQAPRLLIY
DASKRATGIPARFSGSGSGTDFTLTISSLEPEDFAVYYCQQRSKWPPWT
FGQGTKVESKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

In addition to utilizing whole antibodies, the invention encompasses the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

### IGF1R Agonist

In some embodiments, the methods include administration of an IGF1R agonist, e.g., a small molecule agonist, e.g., ginsenoside RG5. RG5 has the following structure:

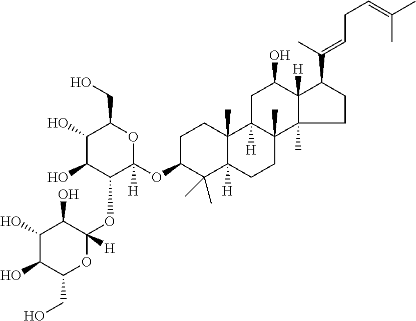

RG5 is commercially available and methods for making RG5 are known in the art (see ref 31).

### Pharmaceutical Compositions and Methods of Administration

The methods described herein include the use of pharmaceutical compositions comprising or consisting of IGF1R agonists, and/or IGF1 and IGF1R antibodies, as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, oral or nasal (e.g., inhalation), transdermal (topical), transmucosal, and ocular administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36(1):49-62 (2008). The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Methods

The following materials and methods were used in the Examples below.

### 1. Animals

Adult (10-12 weeks old) male and female C57BL/6J (B6) (The Jackson Laboratory, 000664), Cx3cr1^{GFP} (The Jackson Laboratory, 005582), DBA/2J mice (The Jackson Laboratory, 000671) Igf1r^{(lax)} inducible knockout mice (The Jackson Laboratory, 012251) as well as Cx₃cr1^{tm2.1(cre/ERT2)} mice were obtained from the Jackson Laboratory. Igf1r^{(lax)} mice were crossed with Cx3cr1^{tm2.1(cre/ERT2)} mice to generated Igf-1r specific knock out on microglia after 3 consecutive days injection of 100 mg/kg tamoxifen to mice. IGFBPL1 deficiency mice at a C57BL/6J genetic background (Knockout Mouse Project Repository, University of California at Davis) were used in following experiments. Mice of both genders were randomized into control and experimental groups. All experimental procedures and use of animals were approved and monitored by the Institute's Animal Care Committee and conform to the standards of the National Institute of Health and the Association for Research in Vision and Ophthalmology.

### 2. Systems Genetic Approach—BXD Retina Array

The Genome-wide transcriptome profiling was taken from 55 BXD strains at their age between 60 and 100 days. Briefly, Mice were sacrificed by rapid cervical dislocation. Retinas were removed immediately and placed in 1 ml of 160 U/ml Ribolock (Thermo Scientific RiboLock RNase #EO038140 U/μl 2500 U) for 1 min at room temperature. The retinas were removed from the eye and placed in Hank's Balanced Salt solution (Gibco, Cat. No. 14175-095) with 50p RiboLock stored in −80° C. The RNA was isolated using a QiaCube and the in column DNase procedure. The samples were analyzed using the Agilent 2100 Bioanalyzer. The samples with RNA integrity values from 7.0 to 10 were run on Affymetrix Mouse Gene 2.0 ST Array at UTHSC. Raw microarray data was normalized using Robust Multichip Array (RMA) method (PMID: 12538238) and then rescaled with 2z+8 (PMID: 15711545), in which, the original non-logged expression estimates were logged and Z normalized. We then multiplied the z score by 2. Finally, we added 8 units to ensure that no values were negative. The normalized expression data can be found at GeneNetwork (www.genenetwork.org) with the accession number of GN709.

In order to identify the covariates associated with Igfbpl1 in the BXD retinas, we performed both genetic and literature correlation analysis on GeneNetwork, in which, genetic correlation was determined by Pearson product correlations value p, while literature correlation determined by r value which described by similar terminology in published papers (PMID: 15308538). Genes with p<0.05 and r>0 were selected for further analysis. Genes with significant correlations with Igfbpl1 (p<0.05 and r>0) were used for gene set over representation analysis for Kyoto Encyclopedia of Genes and Genomes (KEGG) pathway and Mammalian Phenotype Ontology (MPO) on Webgestalt website (http://bioinfo.vanderbilt.edu/webgestalt/) (PMID: 31114916). Mouse genome was used as reference gene set with the minimum of 7 genes for each category. The p values generated from the test were automatically adjusted to account for multiple comparisons using the Benjamini and Hochberg correction (BH) (ref: Benjamini, Y. and Y. Hochberg, Controlling the false discovery rate: a practical and powerful approach to multiple testing. Journal of the Royal statistical society: series B (Methodological), 1995. 57(1): p. 289-300)

### 3. Bulk RNA-Sequencing

We performed RNA-seq using four Igfbpl1−/− mice retina and four WT mice retina. The raw data was quality controlled and aligned on Mus musculus (mouse) reference genome (GRCm38) with STAR v2.5.0a (PMID: 23104886), FeatureCount (v0.6.1) (PMID: 24227677) was used to count the number of read mapped to each gene. Transcripts Per Million (TPM) was calculated for each gene based on the length of the gene and reads mapped to that gene. Differential expression analysis between Igfbpl1^{-/-} and WT mice was performed using the DESeq2 R package (v1.22.2) (PMID: 25516281). gene set over representation analysis for the differentially expressed genes was performed with the same above described method. We also downloaded lists of genes with function related to “Microglia”, “Astroglia”, “neuron”, and “photoreceptor” from genecard (www.genecards.org) with relevance score>5, and determined the percentage of and calculated the proportion of differentially expressed genes in each of those gene lists.

### 4. Immunohistochemistry

For whole mounted retina staining, mouse eyeballs were fixed in 4% paraformaldehyde for 3 hours at room temperature or overnight at 4° C. fridge after sacrificing mice with carbon dioxide (CO₂). Then retinas were dissected out and incubated with blocking buffer (3% donkey serum+1% BSA+0.1% Triton+0.1% Tween in 1×PBS) for 1-3 h RT. Then retinas were subjected to immunolabeling by incubation with primary antibody (Table 1) overnight at 4° C. After washing three times by washing buffer (0.1% Triton-100 in PBS), retinas were incubated with secondary antibody at room temperature for 2 hrs. After rinsing retina three times in washing buffer for 5-10 min on shaker, mount slide with Fluoroshield Mounting Medium with DAPI (Abcam, Cat. No. ab104139). Visualize under microscope (Leica, SP8). Secondary antibody: Streptavidin, Alexa Fluor 546 conjugate (1:1000, S11225, Invitrogen), Streptavidin, Alexa Fluor 488 conjugate (1:1000, S11223, Invitrogen), Cy2 Donkey Anti-rabbit (1:1000, 711-225-152, Jackson ImmunoResearch), Cy3 Donkey Anti-mouse (1:1000, 715-165-150, Jackson ImmunoResearch), Cy3 Donkey Anti-goat (1:1000, 705-165-147, Jackson ImmunoResearch).

For immunostaining on the brain sections, brains were collected after transcardinal perfusion of saline and 4% paraformaldehyde, and them embedded in optimal cutting medium (Tissue Tek, Sakura) and 30 μm thickness of frozen sagittal sections were collected. Wash with PBS with 0.5% Triton for 10 minutes on shaker. Incubate with blocking buffer for 1 h RT (3% donkey serum+0.5% Triton in 1×PBS). Add primary AB (in blocking buffer). Incubate on shaker overnight at 4 C. Rinse in PBS+0.5% Triton for 10 minutes×3 on shaker. Incubate biotin-conjugated anti-rabbit antibody 1:250 (Vector), RT, 4-5 hours. Rinse in PBS on shaker overnight. Incubate in streptavidin conjugated Alexa 488 1:2000 (Invitrogen) for 2 h RT on shaker. Rinse in PBS for 10 min on shaker×3. Incubate with CY3 Donkey anti-mouse 1:1000 (Jackson Immunoresearch) for 1 h RT on shaker. Rinse in PBS for 5-10 min on shaker×3. Mount slide with DAPI. Visualize under microscope (IBA-1 green, pTau red) (Leica, SP8). On each section, 6 areas were randomly captured in cerebellum, cortex and hippocampus. At least 3 sections per brain were imaged. The density, cell body area and the longest process per cell of microglia were averaged per section per sample. All quantification procedures were conducted by 2 investigators in a masked fashion.

| Marker Name | Ratio | Cat. No. | Company |
|---|---|---|---|
| Brn3a | 1:500 | MAB1585 | Millipore |
| IBA-1 | 1:250 in whole-mounted | 019-19741 | Wako |
| | retina; | | |
| | 1:1000 in brain section | | |
| IGFBPL1 | 1:50 | AF4130 | R&D system |
| IGF-1 | 1:50 | AF791 | R&D system |
| IGF-1R | 1:50 | AF-305-NA | R&D system |
| COX2 | 1:500 | MA514568 | ThermoFisher |
| | | | Scientific |
| Pho-Tau | 1:500 | MN1020 | Thermo Fisher |
| | | | Scientific |
| GFAP-Cy3 | 1:400 | C9205 | Sigma Aldrich |

### 5. Purification and Culture of Primary Mouse Microglia Cells and Mouse BV2 Cell Line

i. For Bulk RNA-Seq and Cytokine Array

Retinal microglia isolation started from dissecting retinas out from CX3CR1/GFP mice. The single cell suspension was obtained followed by papain digestion (20 units; Worthington, Lk003150). Tap every 3-5 mins until completely digested and then add equal volume of inhibitor to neutralize the reaction. Then the supernatants were removed by centrifuge at 300 g for 8 mins. Resuspend retinal cell pellets from CX3CR1/GFP mice into cell staining buffer (BioLegend, 420201) with pacific blue anti-mouse CD11b primary antibody (Biolegend, 101224). After incubating 30 mins at 4 degree covered with foil, the cell suspension was washed three times with MACS auto-running buffer (Miltenyi Biotec, Cat. No. 130-091-221) and sent for flow sorting. The cells labeled with both pacific blue and GFP signals were sorted as the retinal microglia using MACS cell sorter. The cells were cultured in fresh DMEM (Thermo Fisher Scientific, 11885084) with supplemented 10% fetal bovine serum (Sigma-Aldrich, F0926-500ML) at 37° C. with 5% C02. Before the following experiments, the medium was changed to fresh medium to remove cellular debris.

ii. For COX2 Immunostaining

For primary retinal microglia isolation, microglia cells were isolated from mice retina using magnetic CD11b microbead (BD, 558013). The purification protocol was following manufacture's instructions. Briefly, the papain dissociated retinal cells were resuspended into diluted BD IMag™ Buffer (BD, Cat. No. 552362). After 6-8 mins washing at 300 g, 50 ul of magnetic CD11b microbeads were added for every 10⁷ total cells and then incubated for 30 mins at 6-12° C. Then bring the BD IMag-particle labeling volume up to 1-8×10⁷ cells/ml with 1×BD IMag™ buffer, and immediately place the tube on the Cell Separation Magnet (BD, Cat. No. 552311). Carefully aspirate off the supernatant after 6-8 minutes incubation at room temperature, and then repeat two times washing. The positive fractions left in the tube were then collected for culture. Around 3000 cells per well were seeded in 8-well Nunc® Lab-Tek™ II Chamber Slide™ (Thermo Scientific, Cat. No. 154534PK). The isolated microglial cells were cultured at 37° C. with 5% C02 in DMEM with 10% FBS and 50 ng/ml rmCSF1 (PeproTech, Cat. No. 315-02).

For primary mouse brain microglia isolation, we optimized our protocol based previously reported protocol². First step was to collect brain after perfusing mice with saline. Then the brain was cut into small pieces and digested in dissociation solution including 1 mg/ml collagenase/dispase (Roche, 11097113001), 80 units of papain (Worthington, Lk003150) and 0.5 mg/ml of Dnase (Worthington, Lk003150) in DMEM without FBS for 45 mins-1 hr at 37° C. After dissociation, the cell suspensions were filtered with 70 um filter (Corning, 431751) and then resuspended in DMEM with 10% FBS and 50 ng/ml rmCSF1 (PeproTech, 315-02). Around 10 millions cells were seeded into one 75 cm2 flask (Corning, 353136). Incubate cells at 37° C. with 5% C02 and change medium every 3-4 days. The microglia purification was conducted when observe cell growth until the pericyte monolayer has formed with microglia on top of it. After washing plates with PBS, 2-3 ml of mild trypsin (0.125% trypsin made up with half volume of stock 0.25% trypsin and half FBS free DMEM) (Thermo Fisher Scientific, 25200056) were added and incubated at 37° C. until observing the dissociation of astrocytes and pericytes. Then the flasks were neutralized and washed with culture medium. After few times of purification step, part of remained cells were collected to test purity by FACS. Left cells were cultured at 37° C. with 5% C02 in DMEM with 10% FBS and 50 ng/ml rmCSF1 for following experiments.

For the purification test of this method, primary brain microglia cultures were incubated with Trypsin-EDTA 0.25% (Thermo Fisher Scientific, 25200056) for 15 minutes resulting cell suspension were incubated with 2.5% BSA with PBS for 15 minutes and then incubated with IBA-1 (019-19741; 1:100; Wako) for 30 min and washed with PBS then cells were incubated with AlexaFlour488 conjugated secondary anti-rabbit (A27034) antibody 1:1,000 in 2.5% NGS. The stained cells populations were recorded using the CyanADP flow cytometer (Beckman Coulter, Brea, CA) and resulting 2D plots (Linear side-scatter vs. log 488 fluorescence) were analyzed using FlowJo (FlowJo LLC, Ashland, OR). The negative population gate was set against microglia cell suspension, incubated for 30 minutes with Goat IgG Isotype Control (1:100, 02-6202; Thermo Fisher Scientific).

iii. For RNA Extraction and qPCR Experiments

For the qPCR experiments, retinal microglia isolation was same as the method used for COX2 immunostaining. Briefly, microglia cells were isolated from each mouse retina using magnetic CD11b microbead (BD, 558013). Following manufacture's instructions. The dissociated retinal cells were resuspended into diluted BD IMag™ Buffer (BD, Cat. No. 552362). After washing, 50 ul of magnetic CD11b microbeads were added for every 10⁷ total cells and then incubated for 30 mins at 6-12° C. Then the tube with cell mixture immediately was placed on the Cell Separation Magnet (BD, Cat. No. 552311). Carefully aspirate off the supernatant after 6-8 minutes incubation at room temperature, and then repeat two times washing. The supernatant contained the negative fraction was saved as the negative control. The positive fractions left in the tube were then collected for RNA extraction and qPCR. The isolated microglial cells were cultured at 37° C. with 5% C02 in DMEM with 10% FBS and 50 ng/ml rmCSF1 (PEPROTECH, Cat. No. 315-02) for following experiments.

iiii. For Western Blotting (WB) Assay

For WB, mouse BV2 cell line was used in this study. It was purchased from ATCC (CRL-2469), and was cultured in DMEM (Thermo Fisher Scientific, Cat. No. 11885084) supplemented with 10% FBS at 37° C. with 5% C02. The medium was changed every 3 days. Cells were then seeded onto 6-well plates and stimulated with LPS (Sigma, Cat #L6529-1MG) for 6 hours and followed by IGFBPL1 (400 ng/ml, R&D system, 4130-BL) and/or IGF1 analog (10 uM, BACHEM, H-1356, Cat. No. 4018631) or NBI-31772 (10 μM, EMD Millipore, Cat. No. 479830). Cells were harvested 48 hrs for following western blotting.

### 6. Real-Time Polymerase Chain Reaction (RT-PCR)

Total RNA was extracted from retinas of adult mice or purified retinal microglial cells using RNeasy Plus Mini kit (Qiagen, 74134) or RNeasy Micro kit (Qiagen, 74004) following manufacturer's instruction. The RNA was reversely transcribed using Takara PrimeScript™ RT Master Mix (Takara Bio, RR036A). A mixture of master mix contained cDNA, 2× Master Mix from KAPA SYBR Fast qPCR kit and 10 mM of specific primers was used to detect specific mRNA expression level using the Mastercycler ep realplex real-time PCR system (Eppendorf, Westbury, NY). The temperature of initial denaturation was set at 95° C. for 2 mins followed by 45 cycles of 15 seconds denaturation (95° C.), 15 seconds annealing (59° C.), and 20 seconds extension (68° C.), and lastly holding at 4° C. Relative amount of specific mRNA transcript was presented in fold changes by normalization to the expression level of the housekeeping gene glyceraldehyde 3-phosphate dehydrogenase (GAPDH). Expression was analyzed using the 2−ΔΔCt method. All primers were synthesized by Integrated DNA Technologies (USA). The sequences of all primers are listed in Table 2.

| Gene | | |
|---|---|---|
| Name | | Sequence |
| Igfbpl1 | Forward | CTGTATGACCCTGGGCAAGT |
| | Reverse | GCCAGACCCAATTACGTGTT |
| Igf1 | Forward | GTGGATGCTCTTCAGTTCGTGTG |
| | Reverse | TCCAGTCTCCTCAGATCACAGC |
| Igf-1r | Forward | CGGGATCTCATCAGCTTCACAG |
| | Reverse | TCCTTGTTCGGAGGCAGGTCTA |
| Cox2 | Forward | GCGACATACTCAAGCAGGAGCA |
| | Reverse | AGTGGTAACCGCTCAGGTGTTG |
| Nox2 | Forward | TGGCGATCTCAGCAAAAGGTGG |
| | Reverse | GTACTGTCCCACCTCCATCTTG |
| CD163 | Forward | GGCTAGACGAAGTCATCTGCAC |
| | Reverse | CTTCGTTGGTCAGCCTCAGAGA |
| Tmem119 | Forward | ACTACCCATCCTCGTTCCCTGA |
| | Reverse | TAGCAGCCAGAATGTCAGCCTG |
| Ccl2 | Forward | CAA CTC TCA CTG AAG CCA G |
| | Reverse | TTA ACT GCA TCT GGC TGA G |
| Cxcl10 | Forward | ATCATCCCTGCGAGCCTATCCT |
| | Reverse | GACCTTTTTTGGCTAAACGCTTTC |
| II-1β | Forward | AAC CTG CTG GTG TGT GAC GTT C |
| | Reverse | CAG CAC GAG GCT TTT TTG TTG T |
| Il-1α | Forward | ACGGCTGAGTTTCAGTGAGACC |
| | Reverse | CACTCTGGTAGGTGTAAGGTGC |
| Iba-1 | Forward | TCTGCCGTCCAAACTTGAAGCC |
| | Reverse | CTCTTCAGCTCTAGGTGGGTCT |
| Ym1 | Forward | TACTCACTTCCACAGGAGCAGG |
| | Reverse | CTCCAGTGTAGCCATCCTTAGG |
| C1q | Forward | GTGGCTGAAGATGTCTGCCGAG |
| | Reverse | TTAAAACCTCGGATACCAGTCCG |
| Tnfa | Forward | TTCTCATTCCTGCTTGTGG |
| | Reverse | TTGGGAACTTCTCATCCCT |
| GAPDH | Forward | CATCACTGCCACCCAGAAGACTG |
| | Reverse | ATGCCAGTGAGCTTCCCGTTCAG |

### 7. Induction of Intraocular Pressure (IOP) Elevation in Mice

Elevated IOP was induced unilaterally in adult mice (10-12 weeks old) as described previously²¹. Briefly, the mice were anesthetized by i.p. injection of ketamine (120 mg/kg)/xylazine (20 mg/kg) mixture using 25-gauge needle. The pupil was dilated with tropical application of 1% Tropicamide followed by proparacaine HCl (0.5%; Baush & Lomb Incorporated, Tampa, FL) to numb the eye. An entry on the corneal was generated by a 30-gauge needle, and a small air bubble was injected into anterior chamber via a glass micropipette which was connected with a Hamilton syringe. 2 μl solution of 10 μm microspheres (9.0×10⁷ beads/ml sterile saline; Thermo Fisher Scientific, Cat. No. F8833) was injected into anterior chamber with care to avoid damaging the iris and the lens. The micropipette will be withdrawn slowly after delivery of microbeads. Antibiotic ointment will be applied topically on the cornea after the injection, and then leave the operated mice to recover on a warm pad and monitored every 15 minutes until awake and fully sternal.

### 8. Proteome Profiler Mouse Cytokine Array

As described above, microglial cells were sorted from CX3CR1/GFP mice as described above. Those isolated microglial cells will be divided into 6 groups: 1. Control; 2. LPS (1 μg/ml, Sigma, Cat #L6529-1MG); 3. IGFBPL1 (400 ng/ml, R&D system, 4130-BL); 4. LPS+IGFBPL1; 5. LPS+IGFBPL1+ IGF1 analog (10 uM, BACHEM, H-1356, Cat. No. 4018631). IGFBPL1 or/and IGF-1R analog were given 6 hours after LPS stimulation. After total 48 hours of incubation, fresh culture medium were collected for cytokine array using Proteome profiler mouse cytokine array Panel A kit (R&D Systems, ARY006). The arrays were conducted according to the manufacturer's protocol as previously described²⁸. The array membranes were imaged with iBright CL1500 imaging system (ThermoFisher Scientific, USA) and the results were analyzed using ImageJ software.

### 9. Genotyping

Mice were anesthetized by isoflurane inhalation (2-4%; Webster Veterinary, Sterling, MA) delivered in 100% Oxygen with a precision Vaporizer. Mouse tail was clipped from each mouse. Care was taken between each mouse to prevent gene contamination in each sample. Then the collected mice tails were lysed using Proteinase K solution (Invitrogen, Cat. No. 25530-049) at 56° C. overnight. After stopping reactions by boiling at 95° C. 30 mins, the mixture of DNA lysates per sample, 2× Hotstar mix (E Enzyme Cat. No DP-008-0250) and specific primer were used for following PCR amplification. DNA size were examined through electrophoresis and then imaged under iBright CL1500 imaging system (ThermoFisher Scientific, USA). All primers were referenced to the information on the website of JAX laboratory and then synthesized by Integrated DNA Technologies (USA).

### 10. Western Blots

WB was performed as previously noted²⁹. Cell or tissue samples were sonicated in a cold RIPA buffer supplemented with a 1:100 FAST protease inhibitor (S8830, Sigma-Aldrich, St Louis, MO, USA). Disruption of the material was performed by a Q55 Sonicator (Qsonica, NY, USA) with four pulses for 22 kHz, 5 s each at 20% power output, and on ice. The lysates were centrifuged at 17,000 for 5 min. The total Protein concentration of resulting supernatants was determined using the Qubit 4 Fluorometer (Thermo Fisher Scientific, Waltham, MA, USA). The 30 μg total protein loaded per lane were separated by SDS-PAGE (4-20% polyacrylamide gel; Biorad) before electrophoretic transfer to 0.45-μm pore nitrocellulose membranes. The membranes were blocked with 2.5% BSA (A7096, Sigma-Aldrich, St Louis, MO, USA) at RT for 1 h and then incubated overnight at 4° C. with the primary antibodies (Table 3). After washing with PBS-T buffer, the blots were incubated with horseradish peroxidase (HRP)-conjugated 1:2000 secondary antibody in 2.5% BSA in PBS-T (Goat anti-Rabbit IgG, 170-6515; Goat anti-Mouse IgG 172-1011) for 1 h at RT. Signals were developed with ECL using a Super Signal West Pico kit (Thermo Fisher Scientific, Waltham, MA, USA) and detected with iBright CL 1500 (Thermo Fisher Scientific, Waltham, MA, USA). The densitometric analysis of WBs was performed with ImageJ software (National Institute of Health, Bethesda, MD, USA) using 8-bit grayscale positive chemiluminescent membrane images. All the quantification results were averaged from three protein blots and expressed as the mean ratio of the values (target protein/housekeeping protein)±standard deviation (SD).

| Marker name | Cat. No. | Company |
|---|---|---|
| Pho-PI3K | 4228S | Cell Signaling Technology |
| Total-PI3K | 11889S | Cell Signaling Technology |
| Pho-Tau | MN1050 | ThermoFisher Scientific |
| Total-Tau | MN1000 | ThermoFisher Scientific |
| β-amyloid | 36-6900 | ThermoFisher Scientific |
| c-Caspase3 | 9661S | Cell Signaling Technology |
| GFAP | 3670S | Cell Signaling Technology |
| β-actin | MA5-15739 | ThermoFisher Scientific |

### 11. Single-Cell RNA Sequencing

Single cell suspensions of retinal CD45⁺ cells taken from Igfbpl1^{-/-} and age- and sex-matched B6 mice that were trans-cardiacally perfused with PBS. Immune cells were stained by PE conjugated anti-mouse CD45 (Biolegend) and sorted by flow cytometry (BD FACSAriaII). Immediately sorted cells (˜20,000 CD45+ cells per sample) were resuspended and washed in 0.05% RNase-free BSA in PBS for single cell library preparation with 10× Chromium Next GEM Single Cell 3′ Kit (10× Genomics according to the manufacturer's instructions. The single cell cDNA libraries were sequenced by NexSeq500 (Illumina). Raw sequences were demultiplexed, aligned, filtered, barcode counting, unique molecular identifier (UMI) counting with Cell Ranger software v3.1 (10× Genomics) to digitalize the expression of each gene for each cell. The analysis was performed using the Seurat 3.0 package (Stuart et al. 2019, Cell). We first processed each individual data set separately prior to combining data from multiple samples. For each data set, we selected the 2,000 most variable genes and also removed outlier cells with very high total UMI counts and high mitochondrial ratio (>10%) from each data set. Subsequently, we ran principal component analysis (PCA) and used the first 15 principal components (PCs) to perform tSNE clustering (resolution=0.3). We checked well-defined marker genes for each cluster to identify potential cell populations, such as T cells (CD3D, CD3E, and CD3G), B cells (CD19, CD20), microglia (CD68, Cx3cr1). For microglia analysis, Cd68 and Cx3cr1 positive clusters were selected for subsequent analyses. We repeated PCA, tSNE clustering (resolution=0.08 on the integrated data sets of microglia. Finally, we performed differential expression tests on the integrated data sets to identify the genes significantly upregulated in each cluster compared with all other cells (adjusted P<0.05) as well as the genes differentially expressed between WT and Igfbpl1−/− cells. For gene sets representing specific cellular functions or pathways, we performed functional enrichment analysis with the biological process of Gene Ontology by the online tool DAVID (http://david.ncifcrf.gov/) (Huang et al. 2007 Genome biology).

### 12. Measurement of IOP

Mice were anesthetized by isoflurane inhalation (2-4%; Webster Veterinary, Sterling, MA) delivered in 100% Oxygen with a precision Vaporizer. The IOP was measured by TonoLab tonometer (Colonial Medical Supply) before and post-MB injection. An average of 6 IOP readings will be taken as one reading. At least 6 readings of IOP level will be taken per mouse eye, and the mean of 3 readings represents the IOP level of a mouse eye. Baseline level of IOP in anesthetized mice was measured before induction of elevated IOP. Then the IOP was measured twice a week after microbeads injection induced IOP elevation. The IOP was measured at the same time in the morning to minimize the circadian variation.

### 13. Intravitreal Injection

Adult mice were anesthetized by i.p. injection of ketamine (120 mg/kg)/xylazine (20 mg/kg) mixture using 25 G needle. The pupil was dilated with tropical application of 1% Tropicamide followed by proparacaine HCl (0.5%; Baush & Lomb Incorporated, Tampa, FL) to numb the eye. A hole on the sclera about 0.5 mm far from the limbus will be punctured using a 30 G needle. 2 μl IGFBPL1 (total 100 ng) or sterile saline was injected into the vitreous via a glass micropipette without damaging the lens or retina. Antibiotic ointment was then applied on the entry site. Mice will be placed on a warm pad and monitored every 15 minutes until awake and fully sternal.

### 14. Electroretinogram (ERG)

The adult mice were dark adapted in a dark adaptation chamber overnight before conducting ERG. Adult mice were anesthetized by i.p. injection of ketamine (120 mg/kg)/xylazine (20 mg/kg) mixture using 25 G needle. Both pupils were dilated by 1% Tropicamide, and a drop of Genteal was applied to keep the corneal moist. The mouse was placed on a warmed platform of ERG machine (Diagnosys LLC). The reference and ground electrodes were inserted beneath skin over forehead and tail, respectively. Two gold-ring recording electrodes were gently placed on the corneas with a drop of artificial tear covered without blocking the pupil. The positive scotopic threshold response (pSTR) were obtained with flash intensities at 6.57E-5[cd·s/m²] and 1.7E-4[cd·s/m²] by averaging 40 responses per intensities. The pSTR was measured from the baseline to the peak of the positive deflection. After the ERG recording, antibiotic ointment and artificial tear were applied to the mice cornea, and mice were left on a warming pad with circulating heated water to maintain body temperature until recover.

### 15. Optomotor Response (OMR)

The mouse was placed on a small platform in the middle of the optomotor chamber. Each trial will begin with a grey homogenous stimulus projected to all screens followed by visual stimuli projected such that a virtual cylinder with rotating gratings is produced. The stimulus will be displayed on all computer monitors using a 4-port video splitter (www.Startech.com, model #ST124PROA), The luminance of individual stripes will be measured using a 371 R Optical Power Meter (Graseby Optronics, Orlando, FL) from the level of the eyes. The contrast at a given spatial frequency is calculated using the formula (Lmax−Lmin)/(Lmax+Lmin) where Lmax is the brightness (in cd/m²) of the white stripe, and Lmin is the brightness of the black stripe (in cd/m²). Contrast level, stripe width, grating speed, and direction of stripe movement (clockwise vs. counterclockwise) were measured. According to the tracking of head movements on the mouse to the moving stripes, the visual acuity and contrast sensitivity can be measured. For example, right eye could detect movement of black and white stripes in anti-clockwise direction, and track well, and vice versa. By changing the stripe width and the contrast, visual acuity and contrast sensitivity of mice can be assessed. Two investigators were required to do the judgement for each step. When both investigators made same agreement, the value can be marked as real value of contrast sensitivity and visual acuity for the mice.

### 16. Electron Microscopy (EM)

The optic nerves were collected after euthanizing mice and placed in 12 Karnovsky's fixative in 0.1M sodium cacodylate buffer for around 24 hours. The optic nerve was placed evenly between two pieces of small filter pad to make sure it straightly. semi-thin section and stained myelin. Using a fine scalpel or precision scissors, trim out central region from transwell membrane sample and place in buffer filled vials into refrigerator. Then, sectioning and staining the myelin of semi-thin at designated 1 um were performed for the optic nerve. The collected sections were followingly imaged under 100× bright field Leica microscope. Quantification of axon numbers was completed by ImageJ with 2 investigators under a masked situation.

### 17. Statistical Analysis

Statistics were analyzed using GraphPad Prism 9 software (GraphPad Inc., La Jolla, CA, USA). Statistical differences among different time points and groups were made by One-way ANOVA and Dunnett's multiple comparisons test. Statistical differences among treatment groups were made by Two-way ANOVA and Tukey's multiple comparisons test. Probability value of P<0.05 were considered to be statistically significant. Data were expressed as mean S.E.M.

### Example 1. Systems Genetics Approach Predicts IGFBPL1 as a Novel Regulator of Microglia

To predict the functional roles and associated signaling cascades of IGFBPL1 in the retina, we employed a systems genetics approach based on genome-wide transcriptome data taken from a murine GRP. Using unsupervised function enrichment analysis of transcriptional profiles of the retinas of more than 50 BXD RI strains (FIG. S1A)^{10, 11}, we projected 127 mouse phenotype ontologies (MPO) for the genes that significantly co-vary with IGFBPL1 expression (FDR<0.01). Interestingly, among the top seven most significant MPO, five were related to inflammatory responses and immune activities. In support of the predicted role of IGFBPL1 in immune modulation, enriched Kyoto encyclopedia of genes and genomes (KEGG) pathway analysis identified cytokine-cytokine and neuroactive ligand-receptor interactions being the top two pathways associated with the function of IGFBPL1 (FDR<0.01). To validate this prediction, we carried out bulk transcriptional profiling using the retinas of Igfbpl1^{-/-} and wild-type C57BL/6J (B6) (WT) mice. Differentially Expressed Genes (DEGs) analysis identified 2,305 transcripts which expression was significantly changed (P<0.05) in retinas of Igfbpl1^{-/-} mice compared to B6 mice. MPO and KEGG analysis predicted similar functional roles of IGFBPL1 in immune modulation and cytokine-cytokine receptor interactions. To further project the type of cells that IGFBPL1 mediates, we next performed retinal cell transcriptional classification and investigated cell-type specific gene expression changes as a result of IGFBPL1 deficiency. We found that the absence of IGFBPL1 induced most robust changes in microglia-related genes compared to all other retinal cell types, including astroglia, photoreceptor, and neurons. These results strongly suggested a role for IGFBPL1 in mediating neuroinflammation, likely through acting on the microglia, in the adult retina.

To explore the possible involvement of IGFBPL1 on microglial function, we performed immunostaining of IGFBPL1 and its signaling receptor, IGF-1R, in normal mouse and human retinas. Expression of IGFBPL1 and IGF-1R in both mouse and human retina were colocalized with IBA-1V retinal microglia. This was further verified by quantitative polymerase chain reaction (qPCR) that Igfbpl1 and Igf-1r mRNA levels were highly expressed in retinal microglial cells that were isolated by magnetic microbeads-conjugated anti-CD11b antibody, compared to other retinal cell types, supporting a possible involvement of IGFBPL1 in microglia.

### Example 2. IGFBPL1 Reverses Lipopolysaccharide (LPS)-Induced Gene Expression and Cytokine Profile Changes in Microglia to a Resting Level Via IGF-1R Signaling

To investigate if IGFBPL1 directly mediates microglial function, we studied gene profiling changes in isolated microglial cultures treated by IGFBPL1. Retinal microglia were purified from mice expressing green fluorescence protein (GFP) driven under a microglial specific promoter Cx3cr1 (Cx3cr1-Gfp) using fluorescence-activated cell sorting (FACS)¹². Purified microglia were cultured in the absence and present of IGFBPL1. In an additional group, we studied the effect of IGFBPL1 on activated microglia. LPS was used to induce an inflammatory response of microglia, and 6 hr later, IGFBPL1 was added. Bulk RNA-seq analysis revealed no different between cultures treated with IGFBPL1 alone and vehicle controls. Remarkably, addition of IGFBPL1 6 hr after LPS stimulation completely reverted LPS-induced microglial gene profile to the control level, suggesting a potent counter-inflammatory effect of IGFBPL1 on retinal microglia. This effects of IGFBPL1 on suppressing microglial activation were verified by cytokine arrays, which measured 36 mouse pro-inflammatory cytokines, chemokines from supernatants of microglial cultures. As described above, the isolated retinal microglia were treated with PBS, LPS, IGFBPL1 alone, or addition of IGFBPL1 at 6 hr after LPS treatment. To further evaluate the involvement of IGF1R in IGFBPL1 signaling, in another group of microglial culture, IGF1 analog which competitively binds IGF1R and functions as an IGF-1R antagonist (IGF-1Ri) were added simultaneously with IGFBPL1 at 6 hr after LPS challenge. As expected, consistent upregulations of pro-inflammatory factors were noticed in LPS-treated group; whereas, addition of IGFBPL1 6 hr later completely reverted the LPS-induced inflammatory cytokines to the control levels, which effects were largely blocked by the presence of IGF1Ri. The data suggest that IGFBPL1 mediates through IGF-1R signaling to drive microglial homeostasis in vitro.

We further corroborated the effect of IGFBPL1 on suppressing microglia activation using the brain microglia and mouse BV-2 microglial cell line. Here we optimized a highly efficient method of microglia purification for the brain or retina. Fluorescence activated cell sorter was used to verify the purity of microglia following isolation, which demonstrated 99.1% purity of IBA-1⁺ cells. Cyclooxygenase 2 (COX2), a biomarker of microglia dependent neuroinflammation, was used as a biomarker of microglial inflammatory response¹³. In all microglial cultures, including those isolated from the retina and brain or the BV2 cells, LPS treatment significantly induced COX2 upregulation, which was suppressed by addition of IGFBPL1 6 hr later. This effect of IGFBPL1 was abolished by the presence of IGF-1Ri.

PI3K pathway was increasingly reported to mediate a broad range of bioactivities on neurodegeneration and neuroinflammation¹⁴. Accordingly, genetic analysis of DEGs between Igfbpl1^{-/-} and WT mouse retinas suggested the involvement of PI3K pathways. Western blot analysis in BV2 microglia showed that while individual treatment of IGFBPL1 or LPS did not alter PI3K signaling, addition of IGFBPL1 6 hr after LPS challenge induced an 8-fold increase in PI3K phosphorylation, and this effect was abolished by the presence of IGF-1Ri. These results show that IGFBPL1 drives microglial homeostasis via IGF-1R/PI3K signaling.

### Example 3. Spontaneous Activation of Microglia in IGFBPL1 Deficiency Mice

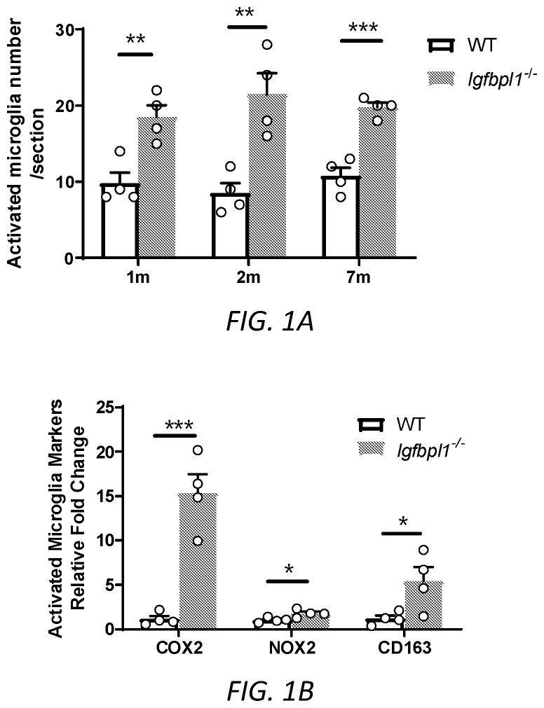

To examine if IGFBPL1 mediates microglial homeostasis in vivo, we performed morphometric and single gene RNA-seq analysis of microglia in Igfbpl1-mice. Microglia is a specialized resident immune cell in CNS, which is responsible for surveilling the CNS microenvironment. Once sensing homeostatic perturbations caused by injuries or diseases, microglia can quickly response and be activated by altering the morphology and producing of proinflammatory cytokines¹⁵. To study the effect of IGFBPL1 in vivo, we quantified the morphological changes of microglia in WT and Igfbpl1^{-/-} mice at the ages of 1-, 2- and 7-months old respectively. IBA-1⁺ microglia from Igfbpl1^{-/-} mice retina exhibited larger cell bodies, shorter dendrites and less ramified cell morphology compared to WT mice at all ages, suggesting chronic inflammation in the retina of Igfbpl1^{-/-} mice. Counts of activated microglia, Igfbpl1^{-/-} mice displayed increased retinal activated microglia number at different ages (FIG. 1A). Results of qPCR using retina from around 7-month-old WT and Igfbpl1^{-/-} mice confirmed significant upregulations of activated microglia markers including Cox2, Nox2 and Cd163 (FIG. 1B) and pro-inflammatory factors, including Ccl2, Cxcl10, II-1β, Il-1α in Igfbpl1^{-/-} mouse retina compared to age-matched WT retinas (FIG. 1C), supporting chronic neuroinflammation in retinas of Igfbpl1^{-/-} mice.

Single cell RNA-seq, a technique with high resolutions of cellular difference and functions, was employed to profile the difference of retinal microglia between WT and Igfbpl1^{-/-} mice retina. The tSNE map of microglia from WT and Igfbpl1^{-/-} mice retina identified retinal microglia into 4 clusters, including cluster 0—resident microglia; cluster 1—Igf1⁺MHCII⁺ inflammatory microglia¹⁶; cluster 2—interferon response microglia (such as Stat1/2, Ifit1/2/3 and/or Cxcl10⁺ microglia); cluster 3, proliferating microglia as Mki67 expressed (FIG. 1D). The dot plots for representative microglia gene markers were presented in the dot plots (FIG. 1D). Cx3cr1, Tmem119, Siglech and Csf1 were identified to be highly expressed in cluster 0 resident microglia cells. The cluster 1 inflammatory microglia were presented as cells expressing Igf1, H2-Aa, Apoe and CD74. The genes of Stat1, Ifit1 and Cxcl10 were seen as interferon (IFN)-response microglia markers, while Mki67 is expressed by cluster 3 proliferating microglia (FIG. 1D). Since the proportion of cluster 1 and 2 microglia in Igfbpl1^{-/-} mice was found increased, it indicated the increased percentage of inflammatory and IFN-response microglia in Igfbpl1^{-/-} mice retina. To further elucidate the involved pathways of cluster 1 and cluster 2 in Igfbpl1^{-/-} mice retina, gene ontology (GO) enrichment analysis was performed. Compared to DEGs in cluster 0 resident microglia, the predicated pathways in cluster 1 inflammatory microglia, including inflammatory response, innate immune response related pathway, was noted to upregulated, while in cluster 2 microglia, the upregulated pathways related to immune response and cellular response to IFN were noticed. When using MacSpectrum to calculate the polarization (M1/M2) index and classified the proinflammatory-proportion, the inflammatory microglia in Igfbpl1^{-/-} mice was 17% and significantly higher than that in WT mice (15%). These all further confirmed that increased inflammation occurred in Igfbpl1^{-/-} mice contributed by the increased proportion of inflammatory and IFN-response microglia.

### Example 4. Spontaneous Neuroinflammation and Progressive Neurodegeneration in the Brain of IGFBPL1 Deficient Mice

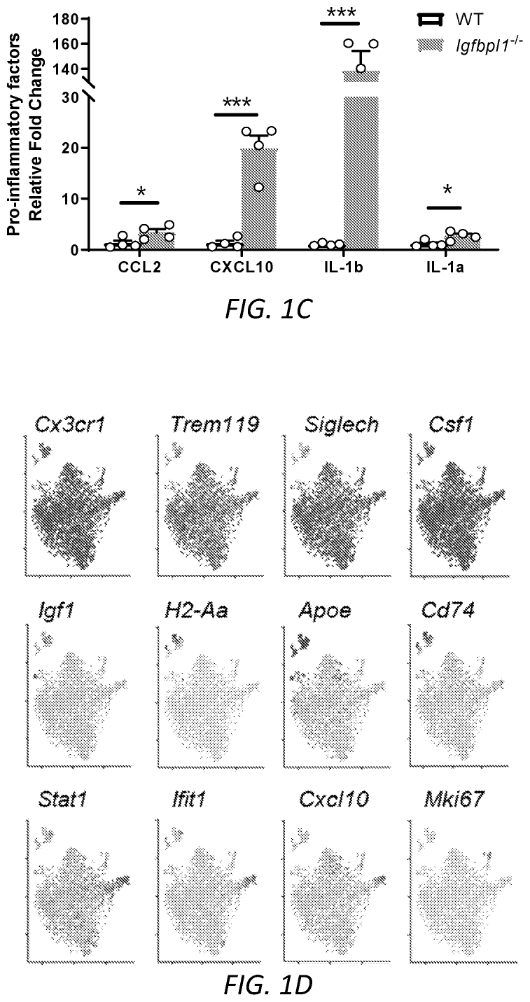

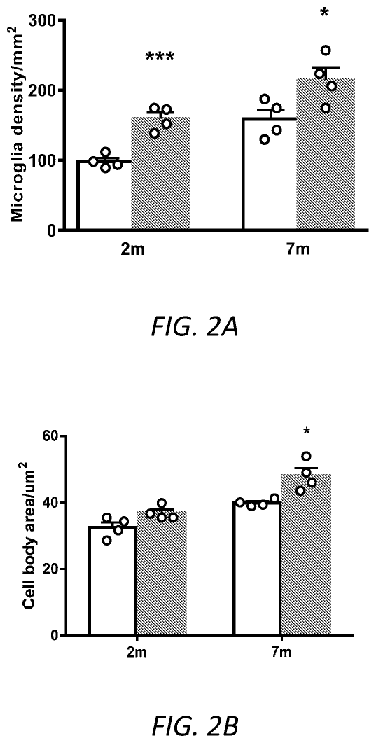

Emerging evidence suggests that retina can be the early biomarker of neurodegeneration in brain^{17, 18}. Because of the identified microglia activation and RGC loss in Igfbpl1^{-/-} mice retina, we next examined if neuroinflammation and neurodegeneration also occurs in the brain of Igfbpl1^{-/-} mice. Remarkably, we noted chronic activation of microglia as shown by increased cell density, enlarged body size and shorten dendrite length in the hippocampus of 2- and 7 m-old Igfbpl1^{-/-} mice, compared WT mice (FIG. 2A-C). To identify if neurodegeneration was induced by microglia activation, phosphorylated tau (pTau) and amyloid beta (Aβ) which are hall markers of Alzheimer's diseases (AD)¹⁹ markers were quantitatively assayed. Significant accumulation of pho-Tau was detected by immunostaining and Western blot in the hippocampus in 7-months old Igfbpl1^{-/-} mice compared to age-matched WT mice (FIG. 3A-E). A similar increase of βamyloid and APP was also noted in the hippocampus of 7-month-old Igfbpl1^{-/-} mice brain, compared to WT mice. c-Caspase 3 activation, a biomarker of apoptosis and neuroinflammation²⁰, was detected in the hippocampus of Igfbpl1^{-/-} mouse brain, and this was confirmed by Western blot analysis. Together, absence of IGFBPL1 leads to chronic neuroinflammation and neural apoptosis in the brain.

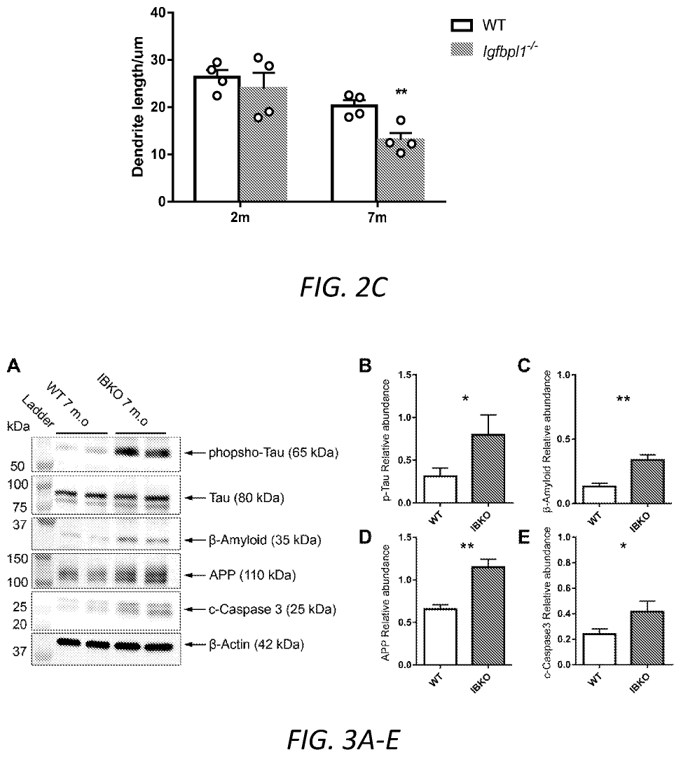

Progressive retinal ganglion cell loss was also detected in the retinas of Igfbpl1^{-/-} mice (FIGS. 4A and B). This phenotype was comparable as glaucoma mice with microglia activation and gradually RGCs loss, eventually caused irreversible vision loss (FIGS. 4C and D). But different to elevated IOP induced neuron loss in glaucoma, the IOP in Igfbpl1^{-/-} mice revealed no difference to age-matched WT mice. These results implied that IGFBPL1 plays a critical role in maintaining microglia homeostasis and subsequently neuron survival through regulating microglia reactivities in both brain and retina.

### Example 5. IGFBPL1 Administration Attenuated Glaucoma Caused Immune Responses in the Retina

To identify if additional IGFBPL1 administration can restore the homeostasis of immune microenvironment in the retina with pathological stress, we employed microbead-induced glaucoma mice model as described above²¹. With the knowledge of microglia activation and pro-inflammatory factors release as the early alteration in glaucoma retina and contributing to the progression of degeneration^{22, 23}, we evaluated microglia activities according to the morphological changes and the production of activated markers after experimental glaucoma model induction in the mice. As described previously²¹, this experimental model is mimicking IOP-dependent glaucoma in clinic. The IOP was monitored twice a week to ensure the success of model building, while saline or IGFBPL1 was intravitreally administrated at day 3 and 10 post glaucoma model building. After 2 wks post glaucoma-induced injury, the retina from vehicle or IGFBPL1 given group mice were used to assess IBA-1⁺ immunolabeled microglia activation. Comparing with the normal retina, microglia in the mice retina with glaucoma possessed shorten process and enlarged cell body and increased cell density, while intravitreally administration of IGFBPL1 can reverse activated microglia into resting state and presented no statistic difference with the baseline profiles (FIGS. 4C and E). In addition to microglia morphology, the expression of activated microglia markers including Iba-1, Ym-1. Nox-2 and Cd163 in the retina were also quantified by qPCR at 14 days post glaucoma induction in mice. Then we found IGFBPL1 treatment can dramatically down-regulate the level of activated microglia markers which was induced by the elevated IOP in glaucoma mice (FIG. 4F). Meanwhile, the expression of pro-inflammatory cytokines in the glaucoma retina, including C1q, Tnfα, Il-1β and Il-1α, was also remarkably reduced by IGFBPL1 given (FIG. 4G).

Additionally, since reactive astrocytes were reported to be neurotoxic to neurons and can be triggered by activated microglia²⁴. We further examined the onset of retinal astrocyte activation through morphology changes and expression level of glial fibrillary acidic protein (GFAP) after experimental glaucoma induction. As expected, elevated IOP led to a significant increase of astrocyte number and GFAP expression in the retina, which was assessed by the immunostaining and GFAP western blot analysis respectively. Remarkably, IGFBPL1 administration significantly restored glaucoma induced reactive astrocyte number and GFAP level at day 14 post glaucoma induction. Collectively, these results suggest that IGFBPL1 treatment effectively suppressed microglia and astrocyte activation and maintained the homeostasis of retinal immune microenvironment.

### Example 6. Therapeutic Administration of IGFBPL1 Prevents Retinal Neuron Loss and Rescues Visual Function in Different Mouse Models of Glaucoma Via Signaling IGF-1R in Microglia

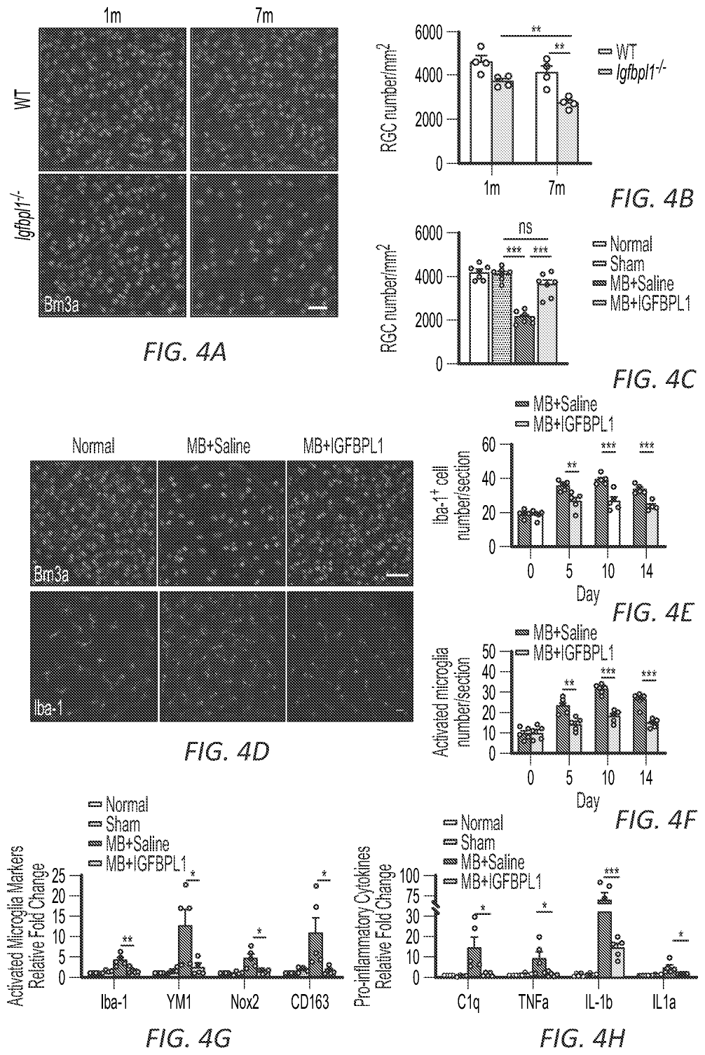

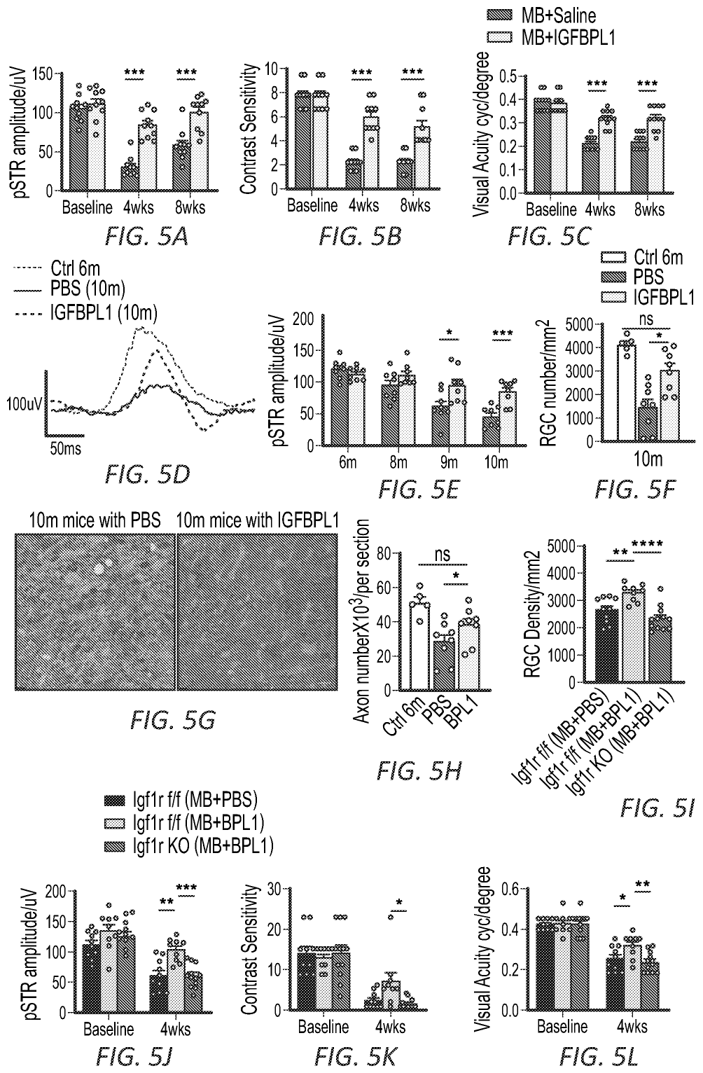

To investigate the therapeutic potential of IGFBPL1 on different mouse models of glaucomatous neuropathy, we examined survived RGCs number and assessed RGC functions in different glaucoma models. Quantification of Brn3a immunolabeled RGCs and positive scotopic threshold response (pSTR) amplitude detected by ERG was applied to assess the effects of neuroprotection²⁵. Correlated with elevated IOP induced by anterior chamber injection of microbeads (FIGS. 6A-H), significant loss of RGCs was identified in MB glaucoma mice with saline intravitreal administration (FIGS. 5C and D). Also, the pSTR amplitude dropped down to around 50% of baseline (FIG. 5A). Surprisingly, IGFBPL1 given intravitreally at day 3, 10 and 17 post IOP elevation significantly prevented RGCs loss and dysfunction from glaucomatous damages (FIGS. 4C and D; FIG. 5A). And both RGC quantification and pSTR amplitude has no significant difference with baseline (FIGS. 4C and D; FIG. 5A). To further determine if enhanced RGC survival and function can improve vision, we assessed mouse contrast sensitivity (CS) and visual acuity (VA) by recruiting optomotor response (OMR)²⁶. Comparing to the significant reduction of CS and VA in MB-induced glaucoma mice with vehicle given, IGFBPL1 treatment restored CS and VA at both 4- and 8-weeks post glaucoma induction (FIGS. 5B and C). Followingly, we examined IGFBPL1 performance on DBA/2J mice which is an animal model with congenital ocular hypertension due to iris pigment dispersion (IPD). Through monitoring the IOP changes in DBA/2J mice, we found that IOP started to gradually increase from the age of 6 m-old (FIG. 6A-H). With the age increase of DBA/2J mice, the progressively loss of RGC survival and function was initiated in vehicle group (FIG. 5D-F). However, IGFBPL1 monthly administration effectively protect RGCs and functions, which were presented as the quantification of RGC density and pSTR amplitude after four doses of IGFBPL1 at the age of 10 m-old DBA2/J mice (FIG. 5D-F; FIG. 6A-H). Besides, the survived axon in optic nerve was also significantly preserved from glaucomatous stress in IGFBPL1 treated group compared to vehicle-treated group (FIGS. 5G and H).

To verify if IGFBPL1 induced neuroprotective effects require IGF-1R involvement, the mice with Igf-1r specific knocked out on microglia was generated by crossing Igf-1r^{f/f} mice with Cx3cr1^{tm2.1(cre/ERT2} mice. The examination of RGC survival number and pSTR function was also applied to study the effects of IGFBPL1 treatment at day 3, 10 and 17 post microbeads-induced glaucoma. Igf-1r^{f/f} mice with glaucoma followed by IGFBPL1 given acted as the positive control, while mice with PBS treatment as negative control. The continuously reduction of pSTR amplitude and RGCs in Igf1r^{-/-} glaucoma mice retina treated with IGFBPL1 suggested the neuroprotective effects diminished after knocking out the IGF-1R on microglia (FIGS. 5I and J). Also, very limited protective capacity of IGFBPL1 was presented on the assessment of CS and VA on Igf-1r^{-/-} mice with MB induced glaucoma (FIGS. 5K and L). The littermate mice with Igf-1r^{f/f} and Igf-1r^{-/-} genotypes were used as the sham control to exclude the influences of any knock-out induced degeneration (FIG. 6A-H). Taken all together, the therapeutic effects of IGFBPL1 on glaucomatous neurodegeneration required the involvement of IGF-1R signaling on microglia.

### Example 7. Anti-Human IGF1R Agonist Antibodies Show a Counter-Inflammatory Effect in LPS-Primed Human Microglial Cell Line

qPCR was used to measure levels of pro-inflammatory cytokines IL-6, IL-1b and IFNa induction in HMC3 microglial cell line that were cultured alone, stimulated by LPS (100 ng/ml) and ATP (5 mM; LPS+ATP) following by treatment with vehicle (PBS), recombinant IGFBPL1 (BPL1) or various IGF1R antibodies at 6 hours after administration of LPS+ATP. The antibodies evaluated are shown in Table 1.

TABLE 1

| Type of | Species | | | Tested Dose in | |
|---|---|---|---|---|---|
| antibody | reactivity | Company | Cat No. | HMC-3 | Immunogen |
| Monoclonal | Mouse anti | R&D | MAB391- | 10 ug/ml | S. |
| Mouse | Human&mouse | systems | 100 | | frugiperda insect |
| IgG1 Clone | | | | | ovarian cell |
| # 33255 | | | | | line Sf 21- |
| (a) | | | | | derived |
| | | | | | recombinant |
| | | | | | human IGF-I |
| | | | | | R/IGF1R Glu31- |
| | | | | | Asn932 |
| | | | | | Accession # |
| | | | | | P08069 |
| Monoclonal | Mouse anti | Novus | NB110- | 1:200 | Purified |
| Ab | Human | Biologicals | 87052 | | recombinant |
| (b) | | | | | fragment of |
| | | | | | IGF1R-Beta |
| | | | | | (AA: 1101- |
| | | | | | 1367) |
| IGF1R | Mouse anti | Abnova | MAB10693 | 1:200 | Recombinant |
| monoclonal | Human | | | | protein |
| antibody, | | | | | corresponding to |
| clone | | | | | human IGF1R. |
| 3G5C1 (c) | | | | | |

Links:
(a) rndsystems.com/products/human-mouse-igf-i-r-igf1r-antibody-33255_mab391
(b) novusbio.com/products/igf-i-r-igf1r-antibody-3g5c1_nb110-87052
(c) abnova.com/products/products_detail.asp?catalog_id=MAB10693

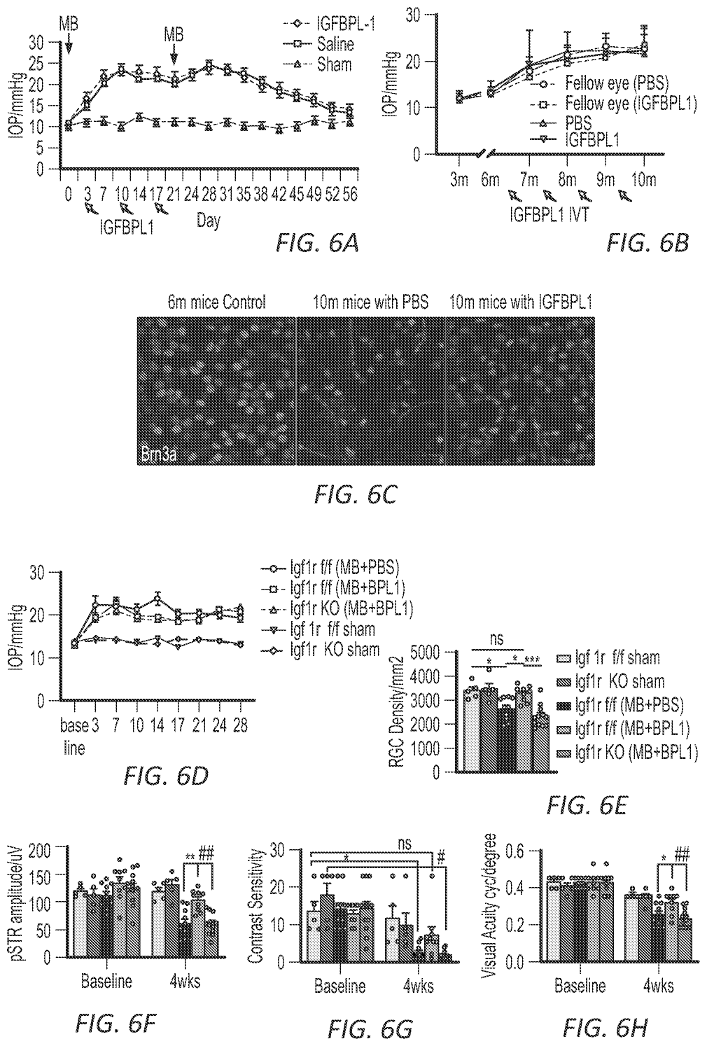

The results, shown in FIG. 7, show a potent anti-inflammatory effect of the Abnova 3 G5C1 anti-IGF1R antibody, but not other (R&D and Novus) antibodies, as assessed by all 3 pro-inflammatory cytokine mRNAs. Error bar=SEM. BPL1 was used as a positive control. *P<0.05 by one way ANOVA (n=4/group).

### Example 8. Screening for Anti-Human IGF1 Agonist Antibody with a Counter-Inflammatory Effect on Microglial Activation

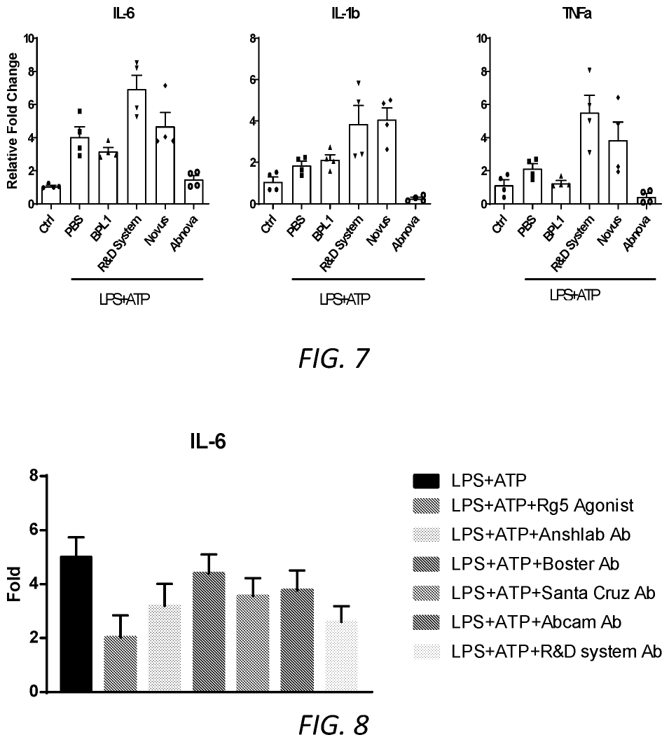

qPCR was used to measure levels of pro-inflammatory cytokine IL-6 induction in a human microglial cell line stimulated by LPS and ATP (LPS+ATP) followed by treatment with IGF1R agonist Rg5 or various IGF1 antibodies 6 hours after LTP+ATP administration. As shown in FIG. 8, IGF1R agonist Rg5 showed the largest anti-inflammatory effect.

### Example 9. Rescue of RGC Function and Vision by Intravitreal Injection of IGF1R Agonist in a Mouse Model of Ischemic Optic Neuropathy

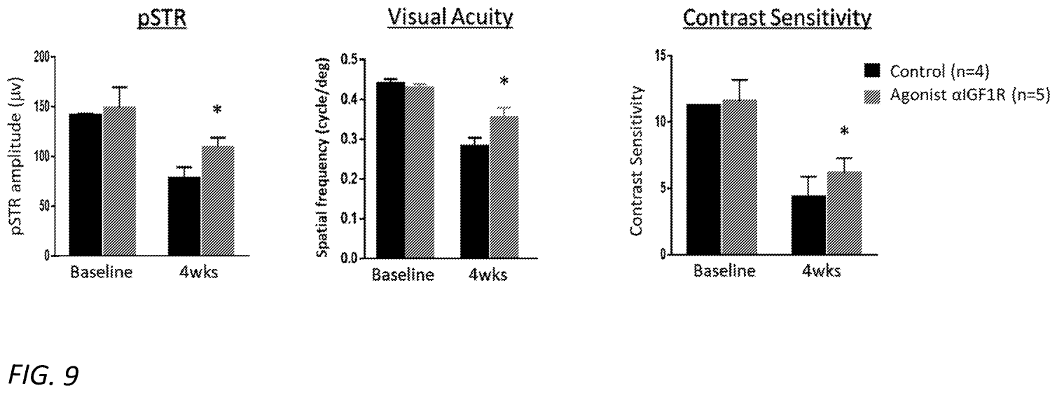

Quantification of electroretinogram positive scotopic threshold response (pSTR) amplitudes, visual acuity and contrast sensitivity was performed before (Baseline) and at 4 weeks (4 wks) after induction of ischemic optic neuropathy in mice received intravitreal injections of control or RG5 IGF1R agonist at days 1, 8, and 17 post injury. The results, shown in FIG. 9, indicate that therapeutic administration of IGF1R agonist by intravitreal injection protects RGC function and vision against ischemic optic neuropathy-induced functional loss.

### REFERENCE

1 Butovsky, O. & Weiner, H. L. Microglial signatures and their role in health and disease. Nat Rev Neurosci 19, 622-635, doi:10.1038/s41583-018-0057-5 (2018).
2 Hammond, T. R., Marsh, S. E. & Stevens, B. Immune Signaling in Neurodegeneration. Immunity 50, 955-974, doi:10.1016/j.immuni.2019.03.016 (2019).
3 Colonna, M. & Butovsky, O. Microglia Function in the Central Nervous System During Health and Neurodegeneration. Annu Rev Immunol 35, 441-468, doi:10.1146/annurev-immunol-051116-052358 (2017).
4 Li, Q. & Barres, B. A. Microglia and macrophages in brain homeostasis and disease. Nat Rev Immunol 18, 225-242, doi:10.1038/nri.2017.125 (2018).
5 Magni, P. et al. Parthenolide inhibits the LPS-induced secretion of IL-6 and TNF-alpha and NF-kappaB nuclear translocation in BV-2 microglia. Phytotherapy research: PTR 26, 1405-1409, doi:10.1002/ptr.3732 (2012).
6 Wei, X. et al. Neuroglobin is an endogenous neuroprotectant for retinal ganglion cells against glaucomatous damage. Am J Pathol 179, 2788-2797, doi:10.1016/j.ajpath.2011.08.015 (2011).
7 Vijaya, L., George, R., Asokan, R., Velumuri, L. & Ramesh, S. V. Prevalence and causes of low vision and blindness in an urban population: The Chennai Glaucoma Study. Indian J Ophthalmol 62, 477-481, doi:10.4103/0301-4738.111186 (2014).
8 Weinreb, R. N., Aung, T. & Medeiros, F. A. The pathophysiology and treatment of glaucoma: a review. Jama 311, 1901-1911, doi:10.1001/jama.2014.3192 (2014).
9 Tian, K., Shibata-Germanos, S., Pahlitzsch, M. & Cordeiro, M. F. J. C. o. Current perspective of neuroprotection and glaucoma. 9, 2109 (2015).
10 Freeman, N. E. et al. Genetic networks in the mouse retina: growth associated protein 43 and phosphatase tensin homolog network. Mol Vis 17, 1355-1372 (2011).
11 Geisert, E. E. et al. Gene expression in the mouse eye: an online resource for genetics using 103 strains of mice. Mol Vis 15, 1730-1763 (2009).
12 Jung, S. et al. Analysis of fractalkine receptor CX(3)CR1 function by targeted deletion and green fluorescent protein reporter gene insertion. Mol Cell Biol 20, 4106-4114, doi:10.1128/mcb.20.11.4106-4114.2000 (2000).
13 Weiwer, M. Exploring COX-2 as a biomarker of microglia-dependent neuroinflammation. Journal of Nuclear Medicine 59, 1628 (2018).
14 Cianciulli, A. et al. Microglia Mediated Neuroinflammation: Focus on PI3K Modulation. Biomolecules 10, 137, doi:10.3390/biom10010137 (2020).
15 Adeluyi, A. et al. Microglia morphology and proinflammatory signaling in the nucleus accumbens during nicotine withdrawal. Science Advances 5, eaax7031, doi:10.1126/sciadv.aax7031 (2019).
16 Ronning, K. E., Karlen, S. J., Miller, E. B. & Burns, M. E. Molecular profiling of resident and infiltrating mononuclear phagocytes during rapid adult retinal degeneration using single-cell RNA sequencing. Scientific reports 9, 4858, doi:10.1038/s41598-019-41141-0 (2019).
17 Normando, E. M. et al. The retina as an early biomarker of neurodegeneration in a rotenone-induced model of Parkinson's disease: evidence for a neuroprotective effect of rosiglitazone in the eye and brain. Acta Neuropathologica Communications 4, 86, doi:10.1186/s40478-016-0346-z (2016).
18 Ngolab, J., Honma, P. & Rissman, R. A. Reflections on the Utility of the Retina as a Biomarker for Alzheimer's Disease: A Literature Review. Neurology and Therapy 8, 57-72, doi:10.1007/s40120-019-00173-4 (2019).
19 Pickett, E. K. et al. Amyloid beta and tau cooperate to cause reversible behavioral and transcriptional deficits in a model of Alzheimer's disease. 29, 3592-3604. e3595 (2019).
20 Płóciennik, A., Prendecki, M., Zuba, E., Siudzinski, M. & Dorszewska, J. J. A. i. A. s. D. Activated caspase-3 and neurodegeneration and synaptic plasticity in Alzheimer's disease. 4, 63 (2015).
21 Chen, H. et al. Optic neuropathy due to microbead-induced elevated intraocular pressure in the mouse. 52, 36-44 (2011).
22 Bosco, A., Steele, M. R. & Vetter, M. L. Early microglia activation in a mouse model of chronic glaucoma. 519, 599-620, doi:10.1002/cne.22516 (2011).
23 Wei, X., Cho, K. S., Thee, E. F., Jager, M. J. & Chen, D. F. J. J. o. n. r. Neuroinflammation and microglia in glaucoma: time for a paradigm shift. 97, 70-76 (2019).
24 Liddelow, S. A. et al. Neurotoxic reactive astrocytes are induced by activated microglia. 541, 481-487 (2017).
25 Pérez de Lara, M. J. et al. Assessment of inner retina dysfunction and progressive ganglion cell loss in a mouse model of glaucoma. Experimental Eye Research 122, 40-49 (2014).
26 Shi, C. et al. Optimization of optomotor response-based visual function assessment in mice. 8, 1-10 (2018).
27 Lian, H., Roy, E. & Zheng, H. Protocol for Primary Microglial Culture Preparation. Bio Protoc 6, e1989, doi:10.21769/BioProtoc.1989 (2016).
28 Tang, Y. et al. Therapeutic Targeting of Retinal Immune Microenvironment With CSF-1 Receptor Antibody Promotes Visual Function Recovery After Ischemic Optic Neuropathy. 11, doi:10.3389/fimmu.2020.585918 (2020).
29 Huang, H. et al. Placental growth factor negatively regulates retinal endothelial cell barrier function through suppression of glucose-6-phosphate dehydrogenase and antioxidant defense systems. FASEB journal: official publication of the Federation of American Societies for Experimental Biology 33, 13695-13709, doi:10.1096/fj.201901353R (2019).
30 Ugradar, S. et al., Teprotumumab for non-inflammatory thyroid eye disease (TED): evidence for increased IGF-1R expression. Eye (Lond). 2020 Nov. 21. doi: 10.1038/s41433-020-01297-w.
31 Kim and Kim, Anti-breast cancer activity of Fine Black ginseng (Panax ginseng Meyer) and ginsenoside Rg5. J Ginseng Res. 2015 April; 39(2):125-34.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a neurodegenerative disease in a subject, the method comprising administering a therapeutically effective amount of an IGF1 or IGF1R antibody or antigen binding fragment thereof, or an IGF1R agonist.

2. A method for treating an immunomodulatory disease in a subject, the method comprising administering a therapeutically effective amount of an IGF1 or IGF1R antibody or antigen binding fragment thereof, or an IGF1R agonist.

3. The method of claim 1, wherein the neurodegenerative disease is Alzheimer's Disease, Parkinson's Disease, amyotrophic lateral sclerosis (ALS), stroke, brain and spinal cord trauma or ischemia, glaucoma, ischemic and traumatic optic neuropathy, Non-arteritic anterior ischemic optic neuropathy (NAION), optic neuritis, age-related macular degeneration, macular edema, or diabetic retinopathy.

4. The method of claim 2, wherein immunomodulatory disease comprises anterior uveitis, dry eye, Sjogren's syndrome, ocular inflammation as a result of ocular disease or procedure, or optionally corneal transplantation.

5. The method of claim 1, comprising administering 3G5C1 antibody or antigen-binding fragments thereof, or a humanized version thereof.

6. The method of claim 1, comprising administering teprotumumab antibody or antigen-binding fragments thereof.

7. The method of claim 1, comprising administering an IGF1R agonist

8. The method of claim 1, wherein the IFG1R agonist comprises a small molecule agonist.

9. The method of claim 1, wherein the IFG1R small molecule agonist comprises RG5.

10. The method of claim 1, wherein the disease is in an eye of the subject, and the method comprises administration to the affected eye.

11.-21. (canceled)
